(19) United States
(12) Patent Application Publication
(10) Pub. No.: US 2004/0077719 A1
(43) Pub. Date: Apr. 22, 2004

(54) Creatine/citric acid compound, method for the production of the same and the use thereof

(76) Inventors: Ralf Jager, Trostberg (DE); Martin Purpura, Bonn (DE); Gunter Ortenburger, Trostberg (DE)

Correspondence Address: Gary M Nath, Nath & Associates, Sixth Floor, 1030 15th Street N W, Washington, DC, 20005, US

(21) Appl. No.: 10/465,917

(22) PCT Filed: Nov. 16, 2001
(86) PCT No.: PCT/EP01/13332

(30) Foreign Application Priority Data
Dec. 28, 2000 (DE) 100 65478.9

(51) Int. Cl. A61K 31/205

(52) U.S. Cl. 514/554

(57) ABSTRACT

The invention relates to addition and/or co-ordination compounds consisting of creatine and citric acid in the ratio of >1.0 to 3.0:1.0. Said compounds are characterised in that they have (KBr) bands in the IR spectrum, where ν=3425 (±5) cm⁻¹ (s), 1624 (±5) cm⁻¹ (m-s) and 1247 (±5) cm⁻¹ (m). Said compounds, which preferably contain no organic solvent and are present in a solid, pulverulent form are produced in particular by a method, according to which, a₁) creatine, containing a maximum content of 20 wt. % water, is reacted with citric acid or its salts at temperatures between 0 and 70° C., or a₂) creatine and citric acid or its salts, containing a total water content of between 5 and 15 wt. %, are ground at temperatures between 0 and 70° C., or a₃) creatine is reacted with citric acid or its salts, in the presence of a solvent at temperatures between 0 and 70° C. The corresponding solvent is then eliminated to a maximum residual content of 1.0 wt. %. The invention also relates to formulations that are suitable for use as food supplements, feed additives, dietetic agents, in functional foods, as neuroprotective agents, in addition to in the cosmetics industry etc.

The present invention relates to creatine/citric acid compounds, the production thereof and suitable formulations as well as the corresponding use thereof.

Creatine is primarily found in muscle and nerve tissue and represents, in its resultant metabolite form, i.e. phosphocreatine, an important energy reserve of the muscle and the brain.

In nerve and heart muscle tissue, creatine appears to have a prophylactic and therapeutic effect in cases of ischaemia caused, for example, by infarction processes or by prenatal and perinatal oxygen deficiency.

Creatine has been known as a muscular substance for over 100 years and acts as an energy source for the muscle.

As an endogenous substance, creatine is not only a valuable dietary supplement, but also has valuable therapeutic properties. It has been shown in a number of scientific works that the controlled intake of creatine over a long period of time can lead to a clear increase in muscle mass and muscle performance as well as in strength and stamina.

A prophylactic, therapeutic or dietetic application of creatine using the most diverse forms of administration (e.g. oral, intravenous, etc.) requires high bioavailability and thus good solubility in water. Creatine, however, which is an amino acid derivative in the form of an internal salt, does not generally possess this property to a sufficient extent, which is why creatine mostly achieves its undisputed advantages, above all in the areas of supplementation and therapies, by means of one of its physiologically compatible creatine salts.

Citric acid is a tricarboxylic acid which is common in the plant kingdom and in many animal tissues and bodily fluids. Citric acid is inter alia one of the number of prominent compounds which participate in the physiological oxidation of fats, proteins and carbohydrates to carbon dioxide and water (cf. inter alia the tricarboxylic acid cycle).

Citric acid, which was isolated from lemon juice for the first time in 1784 by the Swedish chemist Karl Wilhelm Scheele, is currently produced using biotechnology from the fermentation of cane sugar or molasses by the fungus Aspergillus niger. Citric acid is used in its natural form or as the food additive E 330 in the production of foods and also in baking powders, effervescent soda drinks and in general for acidification and as a flavouring agent. Citric acid furthermore helps to increase the stability of foods or other organic substances.

Pure, anhydrous citric acid mostly forms colourless, rhombic crystals having a slightly sour taste.

Known from the U.S. Pat. No. 5,973,199 are inter alia isolated, water-soluble creatine and citric acid salts having a melting point of 112 to 114° C. This creatine citrate is produced by suspending citric acid monohydrate in ethyl acetate, subsequently adding creatine monohydrate and finally mixing the mixture for 4 hours at 25° C. A product containing approximately 90% creatine citrate having a melting point of between 112 and 114° C. and varying solvent parts is hereby obtained.

U.S. Pat. No. 6,211,407 (published on Apr. 3, 2001) describes dicreatine and tricreatine citrates, the melting points of which are supposed to be 146 and 154° C. and which have a creatine content of 57.7 and 67.2 wt-%. Details regarding the structure of such salts and proof for the existence thereof cannot, however, be seen from U.S. Pat. No. 6,211,407.

Effervescent formulations containing creatine citrate as 1:1 salt or physical mixtures of creatine and citric acid are known from the patent specification U.S. Pat. No. 5,925,378.

The object of the present invention was to develop compounds consisting of creatine and citric acid which are particularly physiologically valuable, simultaneously have good water solubility and sufficient storage stability, and which can be produced in a simple manner.

This object is solved by addition or complex compounds consisting of creatine and citric acid in the ratio of >1.0 to 3.0:1.0, which are characterised by an IR spectrum (KBr) where ν=3425 (±5) cm⁻¹ (s), 1624 (±5) cm⁻¹ (m-s) and 1247 (±5) cm⁻¹ (m).

It was surprisingly shown for these compounds that the creatine/citric acid compounds according to the invention have a good storage stability even though the creatine salts known hitherto degrade easily to form creatinines. Furthermore, it was assumed that pure salts are obtained, which can, however, be ruled out inter alia owing to the IR bands and to the presence of simple physical mixtures.

The addition and/or complex compounds are characterised in particular in that they have a melting point which is between 110 and 160° C., and that they are generally free of organic solvents, containing a maximum of 0.1 wt-% thereof, which additionally increases the quality of the product characteristics.

Furthermore, the present invention takes into consideration that the addition and/or complex compounds consisting of creatine and citric acid are in a solid state, with powdered variants having proven themselves to be particularly suitable.

In addition to the compounds themselves, the present invention also claims a method for the production thereof, wherein in three alternative first steps either,

a₁) creatine having a maximum content of 20 wt-% water, in particular 10 to 15 wt-% water, is reacted with citric acid monohydrate and/or anhydrous citric acid at a temperature of between 0 and 70° C., in particular between 10 and 25° C., or

a₂) creatine and citric acid having a total water content of 5 to 15 wt-% are mixed without a solvent or dispersing agent at a temperature of between 0 and 70° C., in particular between 10 and 25° C., and are then reacted by means of grinding in a mill, preferably in an impact mill or another suitable grinding means;

or alternatively

a₃) creatine is reacted with citric acid monohydrate and/or anhydrous citric acid in an aqueous or alcoholic solution or in the presence of a solvent which cannot be mixed with water, preferably ethyl acetate, at a temperature of between 0 and 70° C.

The citric acid components can also be alternatively or additionally used herein in the form of suitable citric acid salts.

Following each of the three alternative method steps a,) to a₃) as cited above, in the method step below,

b) the water or solvent is subsequently removed, preferably under vacuum, to leave a maximum residual content of 5 wt-%.

In addition to the compounds according to the invention, it is obviously also possible with each of these method variants to produce the 1:1 salt of creatine citrate.

In addition to the compounds themselves and the specified method of production, the present invention also claims stable, physiologically compatible formulations which contain the addition and/or complex compound consisting of creatine and citric acid, with formulations containing further physiologically compatible and/or physiologically active additives and/or formulation agents being particularly preferred.

Sugar, alcohols, vitamins, trace elements, amino acids, neurotransmitters, stimulants as well as colouring and flavouring agents have proven suitable from the number of particularly suitable physiologically compatible and/or physiologically active additives. Preferred formulation agents are carbohydrates, such as, for example, methylcelluloses, SiO₂, dissolving agents, flavouring agents, stearates as well as other release agents and preservatives and texturants.

Finally, the present invention also includes the use of the addition and/or complex compound as a dietary supplement, as an animal food additive, as dietetic agents, in functional foods, for special nutrition and to increase strength and endurance in the field of sport; the compounds according to the invention can also be used for rehabilitation, as neuroprotective agents, for improving wound healing, in cases of myopathy and dystrophy and in the field of cosmetics, which the invention also takes into consideration.

A qualitatively outstanding product which is, in general, largely free from organic solvents, which is extremely stable in storage owing to its low water content and which, in spite of its non-salt-like character, still has valuable physiological properties is obtained with the additive and/or complex compounds consisting of creatine and citric acid as claimed above.

The following examples clarify these advantages of the additive and/or complex compound according to the invention.

### EXAMPLES

### Example 1

Method for the Production of a Creatine/Citric Acid Compound Having a Creatine/Citric Acid Composition in a Molar Ratio of 3:1

448 kg (3 mol) of creatine monohydrate and 4.5 kg (0.03 mol) of water were placed in a drier and mixed for half an hour. 210 kg (1 mol) of citric acid monohydrate was subsequently added thereto. After an hour, the reaction mixture was mixed for a further hour under vacuum. The temperature was increased for drying to a maximum of 60° C. by means of a dosed input of heat. Approximately 593 kg (>99%) of a creatine/citric acid composition having a residual moisture content of 0.04 wt-% (according to the Karl-Fischer method) were obtained in this manner.

Melting point: 148-152° C.;

IR (KBr): ν=3425 cm⁻¹ (s), 1663 (s), 1624 (s) and 1247 (m).

### Example 2

Method for the Production of a Creatine/Citric Acid Compound Having a Creatine/Citric Acid Composition in a Molar Ratio of 2:1

301 kg (2 mol) of moist creatine is obtained from the moist creatine (approximately 20 to 25% water) originating from the method for producing creatine monohydrate according to EP-B 754 679 by means of drying until a residual content of 14% water is obtained. 210 kg (1 mol) of citric acid monohydrate was subsequently added thereto. After an hour, the mixture was mixed for a further hour under vacuum. The temperature was increased for drying to a maximum of 60° C. by means of a heat input. Approximately 457 kg (>99%) of a creatine/citric acid composition having a residual moisture content of 0.07 wt-% (according to the Karl-Fischer method) were obtained in this manner.

Melting point: 145-150° C.;

IR (KBr): ν=3425 cm⁻¹ (s), 1669 (m), 1624 (s) and 1247 (m).

### Example 3

Method for the Production of a Creatine/Citric Acid Compound Having a Creatine/Citric Acid Composition in a Molar Ratio of 1.5:1

2.24 kg (0.015 mol) of creatine monohydrate and 2.10 kg (0.01 mol) of citric acid monohydrate were mixed at room temperature and subsequently reacted in an impact mill (99%<150 μm) by grinding. The product was then dried. Approximately 3.92 kg (>99%) of a creatine/citric acid composition having a residual moisture content of 0.06 wt-% (according to the Karl-Fischer method) were obtained in this manner.

Melting point: 130-138° C.;

IR (KBr): ν=3428 cm⁻¹ (s), 1700 (m), 1619 (s) and 1247 (m).

### Example 4

Method for the Production of a Creatine/Citric Acid Compound Having a Creatine/Citric Acid Composition in a Molar Ratio of 3:1

448 kg (3 mol) of creatine monohydrate and 500 kg of ethanol were placed in a drier and mixed for half an hour. 210 kg (1 mol) of citric acid monohydrate was subsequently added thereto. After four hours, the reaction mixture was mixed for a further hour under vacuum. The temperature was increased for drying to a maximum of 60° C. by means of a dosed input of heat. Approximately 593 kg (>99%) of a creatine/citric acid composition having an ethanol residual content of 0.1 wt-% (gas-chromatographic) were obtained in this manner.

Melting point: 148-152° C.;

IR (KBr): ν=3425 cm⁻¹ (s), 1663 (s), 1624 (s) and 1247 (m).

The creatinine content in all of the four examples of the invention was below the detection limit, i.e. below 100 ppm.

1. An addition and/or complex compound consisting of creatine and citric acid in the ratio of >1.0 to 3.0:1.0, characterised by an IR spectrum (KBr) of ν=3425 (±5) cm⁻¹ (s), 1624 (±5) cm⁻¹ (m-s) and 1247 (±5) cm⁻¹ (m), and in that said compound contains a maximum of 0.1 wt-% of organic solvent.

2. A compound according to claim 1, characterised in that said compound has a melting point of 110 to 160° C.

3. A compound according to one of claims 1 or 2, characterised in that said compund is in a solid, preferably powdered state.

4. A method for the production of the compound according to one of claims 1 to 3, characterised in that
a₁) creatine having a maximum content of 20 wt-% water, in particular 10 to 15 wt-% water, is reacted with citric acid monohydrate and/or anhydrous citric acid and/or suitable salts thereof at a temperature of between 0 and 70° C., in particular between 10 and 25° C., or
a₂) creatine and citric acid and/or suitable salts thereof having a total water content of 5 to 15 wt-% are mixed without a solvent or dispersing agent at a temperature of between 0 and 70° C., in particular between 10 and 25° C., and are then reacted by means of grinding in a mill, preferably in an impact mill; or
a₃) creatine is reacted with citric acid monohydrate and/or anhydrous citric acid and/or suitable sorts thereof in an aqueous or alcoholic solution or in the presence of a solvent which cannot be mixed with water, preferably ethyl acetate, at a temperature of between 0 and 70° C., and
b) the water or solvent is subsequently removed, preferably under vacuum, to leave a maximum residual content of 0.1 wt-%.

5. A formulation containing the compound according to one of claims 1 to 3.

6. A formulation according to claim 5, characterised in that said formulation contains further physiologically compatible and/or physiologically active additives and/or formulation agents.

7. A formulation according to one of claims 5 or 6, characterised in that said formulation contains sugar, alcohols, vitamins, trace elements, amino acids, neurotransmitters, stimulants as well as colouring and flavouring agents as the physiologically compatible and/or physiologically active additives.

8. A formulation according to one of claims 5 to 7, characterised in that said formulation contains carbohydrates, SiO₂, stearates, dissolving agents, flavouring agents, preservatives and release agents as well as texturants as formulation agents.

9. Use of the compound according to claims 1 to 3 as a dietary supplement, as an animal food additive, as dietetic agents, in functional foods, for special nutrition, to increase strength and endurance in the field of sport, for rehabilitation, as neuroprotective agents, for improving wound healing, in cases of myopathy and dystrophy and in the field of cosmetics.
